(19) United States
(12) Patent Application Publication
(10) Pub. No.: US 2007/0185444 A1
(43) Pub. Date: Aug. 9, 2007

(54) Vascular clip-on occlusion system

(75) Inventors: Charles Louis Euteneuer, St. Michael, MN (US); Thomas R. Hektner, Medina, MN (US)

Correspondence Address: Thomas R. Hecktner;Starfire Medical, Inc., Suite 100, 15350 - 25th Avenue North, Plymouth, MN, 55447-2082, US

(73) Assignee: Starfire Medical, Inc.

(21) Appl. No.: 11/347,166

(22) Filed: Feb. 3, 2006

(51) Int. Cl. A61M 29/00

(52) U.S. Cl. 604/96.01; 606/194

(57) ABSTRACT

An occlusion system is disclosed for use with a catheter having a catheter proximal end and a catheter distal end. The catheter further has a catheter wall with an outer catheter dimension at the catheter distal end and with the catheter wall defining a catheter lumen with a longitudinal axis extending from the catheter proximal end and through the catheter distal end. The occlusion system includes an occlusion member having an inner wall and an outer wall. The inner wall defines an occlusion member lumen extending throughout an axial length of the occlusion member. The outer wall is expandable between a contracted state with a first transverse dimension and an expanded state with a second transverse dimension. The second transverse dimension is greater than the outer catheter dimension. The occlusion member is sized for the occlusion member to be received at least partially around an exterior of the catheter lumen with the catheter received within the occlusion member lumen and advanced from the catheter proximal end toward the catheter distal end.

### CROSS-REFERENCE TO RELATED APPLICATIONS

This application discloses and claims material disclosed in commonly assigned U.S. patent applications Ser. Nos. ______, ______ and ______, filed concurrently herewith in the names of the same inventors as the present application and respectively entitled “VASCULAR OCCLUSION DELIVERY”, “RAPID BALLOON COUPLING SYSTEM” and “VASCULAR THERAPY DELIVERY SYSTEM” and assigned attorney docket numbers 15059.1US01, 15059.3US01 and 15059.4US01 respectively.

### BACKGROUND OF THE INVENTION

1. Field of the Invention

This invention pertains to occlusion systems for use in vascular applications. More particularly, this invention pertains to endovascular devices which can be rapidly deployed during a catheter-based procedure.

2. Description of the Prior Art

Vascular catheters are well known for accessing treatment sites within a patient's vascular system. Examples of catheter systems can be found, for example, in U.S. Pat. No. 5,830,183 to Krieger dated Nov. 3, 1998, U.S. Patent Application Publication No. U.S. 2004/0143286A1 to Johnson et al., published Jul. 22, 2004, U.S. Patent Application Publication No. U.S. 2005/0059990A1 to Ayala et al., dated Mar. 17, 2005 and U.S. Patent Application Publication No. U.S. 2005/0107819A1 to Sater, published May 19, 2005. Other examples include U.S. Pat. No. 6,168,586 to Hahnen dated Jan. 2, 2001 and U.S. Pat. No. 6,679,871 to Hahnen dated Jan. 20, 2004.

In addition to widespread use in coronary and peripheral arteries, catheter-based therapies are becoming increasingly frequent in neuro-interventional applications. For example, catheters may be used in neuro-interventional applications for delivery of occlusion devices to aneurysms in the brain. Such occlusion devices may include detachable balloons or coils to be placed in an aneurysm. Catheters may also be used for drug delivery to localized areas within the brain.

Historically, catheters would be placed within the vasculature of the brain (e.g., a cerebral artery) by first advancing a guide wire under fluoroscopy through the vasculature and then passing a guiding catheter over the guide wire. Recently, a new generation of stiffer, pre-formed guiding catheters has been introduced for neuro-endovascular applications. A summary of clinical experience with such catheters is described in Putman et al., “Use of Large-Caliber Coronary Guiding Catheters for Neuro-Interventional Applications”, American Journal of Neuro Radiology, pp 697-704 (April 1996).

In addition to guide catheters, so-called micro catheters have also been used in neuro-interventional applications. Commonly, a guide catheter is advanced through the carotid artery until the distal tip is advanced to an optimal penetration location. A micro catheter is advanced over a guide wire through a guide catheter and beyond the distal tip to access narrower vessels with a more tortuous path. Guide catheters are commonly polymer material over metallic braiding with several stiffness zones and a soft distal tip. Micro catheters are commonly formed of braid or coiled materials coated with or alternatively, over-extruded with polymers. It will be appreciated that the invention of the present application is applicable to guide catheters, micro catheters as well as other therapeutic or diagnostic catheters and catheter delivery systems. These endovascular catheters can make it possible to deliver or perform a variety of therapeutic and diagnostic functions within the flow lumen. Examples include: occlusion of flow, measurement of physiologic pressures, removal of tissues, compaction or other alterations of tissue, deposit of permanent implants such as embolic particles, glues, coils, stents and balloons, dissolution of clot and delivery of drugs and other agents or injection of contrast media.

When advancing a catheter through the neuro-vascular system, additional risks are encountered over those associated with advancing a catheter through coronary or peripheral vessels. Within the brain, much of the distal vasculature is typically one to four millimeters in diameter with thin fragile walls. Their profile, shape and flow paths, as viewed with conventional imaging technologies such as fluoroscopy, computer topography and magnetic resonance imaging reveals a complex network with many turns and twists creating a tortuous path. Further, unlike the coronary vessels or peripheral vessels, vasculature within the brain is not supported and reinforced by muscular tissue. Instead, the fragile blood vessels in the brain are typically surrounded by fluid or soft tissue making them more susceptible to risk of tearing or perforation.

In the event a blood vessel within the brain is perforated during advancement of a catheter, it is desirable to rapidly isolate the perforation through occlusion or other techniques to prevent serious adverse consequences of blood flowing from the perforated vessel into the brain. However, providing such an occlusion mechanism can take an unacceptable length of time even under the care and direction of highly experienced interventional radiologists or other health care providers.

It is an object of the present invention to provide an endovascular catheter delivery system which can be used in conjunction with a pre-placed catheter.

It is another object of the present invention to provide a catheter that is designed to enable optimal therapy delivery that exploits the physical position and stability of a previously positioned catheter, that together, provide a superior therapeutic outcome than the original catheter can provide in and of itself. In other words, this system of catheters provides a therapeutic delivery system that utilizes the shaft of the first catheter, pre-positioned in place within the vasculature to navigate or track, over it, or through it, a second catheter that supplements the therapy with additional therapeutic advantage.

It is another object of this invention to provide the physician a secondary or backup device that can be advanced, if desired, over the primary device in order to expand the possible combination of therapies that can be advanced to the targeted therapeutic site.

It is another object of this invention to provide the physician with the option of offering combinations of several possible therapies: occlusion of flow, measurement of physiologic pressures, removal of tissues, compaction or other alterations of tissue, deposit of permanent implants such as embolic particles, glues, coils, stents and balloons, dissolution of clot and delivery of drugs and other agents. One therapy may be offered on the primary catheter and other may be offered on the secondary catheter.

It is another object of this invention to provide rapid, accurate delivery of additional therapeutic value.

It is another object of this invention to provide the physician/catheter operator additional and optional therapies to the patient

### SUMMARY OF THE INVENTION

According to a preferred embodiment of the present invention, an occlusion system is disclosed for use with a catheter having a catheter proximal end and a catheter distal end. The catheter further has a catheter wall with an outer catheter dimension at the catheter distal end and with the catheter wall defining a catheter lumen with a longitudinal axis extending from the catheter proximal end and through the catheter distal end. The occlusion system includes an occlusion member having an inner wall and an outer wall. The inner wall defines an occlusion member lumen extending throughout an axial length of the occlusion member. The outer wall is expandable between a contracted state with a first transverse dimension and an expanded state with a second transverse dimension. The second transverse dimension is greater than the outer catheter dimension. The occlusion member is sized for the occlusion member to be received at least partially around an exterior of the catheter lumen with the catheter received within the occlusion member lumen and advanced from the catheter proximal end toward the catheter distal end.

### BRIEF DESCRIPTION OF THE DRAWINGS

FIG. 1 is a plan view of a rapid delivery occlusion system according to a preferred embodiment of the present invention and shown in exploded format relative to a prior art catheter;

FIG. 2 is a cross sectional view of an occlusion member of the occlusion system of FIG. 1 shown in a contracted state;

FIG. 3 is the view of FIG. 2 showing the occlusion member in an expanded state and prior to inflation of a balloon;

FIG. 4 is the view of FIG. 3 following inflation of a balloon;

FIG. 5 illustrates advancement of the occlusion system of FIG. 1 with the occlusion member in the state of FIG. 2 being advanced through a guide catheter residing within a blood vessel;

FIG. 6 is the view of FIG. 1 following advancement of the occlusion member beyond a distal tip of the guide catheter and with the occlusion member shown in the state of FIG. 3;

FIG. 7 is the view of FIG. 6 with the occlusion member retracted onto a distal tip of the catheter and with occlusion member in the state of FIG. 3;

FIG. 8 is the view of the FIG. 7 with the occlusion member inflated to the state of FIG. 4;

FIG. 9 is a perspective view of a rapid delivery occlusion system according to a second embodiment of the present invention;

FIG. 9A is a schematic representation of a blood vessel with branches and showing a microcatheter with a distal tip in a branch;

FIG. 9B is the view of FIG. 9A with an occlusion provided proximal to the distal tip of the microcatheter;

FIG. 10 is a perspective view of a distal end of the occlusion system of FIG. 9;

FIG. 11 is a view taken along line 11-11 of FIG. 10;

FIG. 12 is a view taken along line 12-12 of FIG. 10;

FIG. 12A is a view of FIG. 12 with a balloon element inflated;

FIG. 13 is a plan view of a third alternative embodiment of a rapid delivery occlusion system;

FIG. 14 is a side elevation view, taken partially in section and in exploded format of a still further alternative embodiment of a rapid delivery occlusion system;

FIG. 15 is a plan view of the occlusion system of FIG. 14 in a sterile package;

FIG. 16 is a side sectional view of a further embodiment of the present invention in place on a micro catheter;

FIG. 16A is the view of FIG. 16 showing an alternative embodiment;

FIG. 17 is a view taken along line 17-17 of FIG. 16;

FIG. 18 is a view taken along line 18-18 of FIG. 16;

FIG. 19 is an end view, partially in section, of a shuttle member carried on a micro catheter shaft;

FIG. 20 is a perspective view of the shuttle of FIG. 19 and showing a micro catheter shaft in phantom lines;

FIG. 21 is a side elevation view of a still further embodiment of the present invention;

FIG. 22 is a view taken along line 22-22 of FIG. 21;

FIG. 22A is a view taken along line 22A-22A of FIG. 21;

FIG. 23 is a side sectional view of a distal end of the apparatus of FIG. 21;

FIG. 24 is a side elevation view of the still further embodiment of the present invention;

FIG. 25 is a view taken along line 25-25 of FIG. 24;

FIG. 25A is a view taken along line 25A-25A of FIG. 24;

FIG. 26 is a side elevation view shown partly in section of a still further embodiment of the present invention; and

FIG. 27 is a view taken along line 27-27 of FIG. 26 and additionally showing an internal guide catheter.

### DETAILED DESCRIPTION OF THE PREFERRED EMBODIMENT

With reference to the various drawing figures in which identical elements are numbered identically throughout, a description of a preferred embodiment of the present invention will now be provided.

Throughout, description may be made of certain selected materials which may be used in a preferred embodiment of the present invention. However, it will be appreciated that material selection may vary as will occur to one of ordinary skill in the art. It is intended that all materials used within the apparatus of the present invention shall be biocompatible materials selected for safe use within human vasculature and susceptible to withstanding the rigors of sterilization procedures in accordance with regulations of the United States Food and Drug Administration or similar regulatory authorities in other countries.

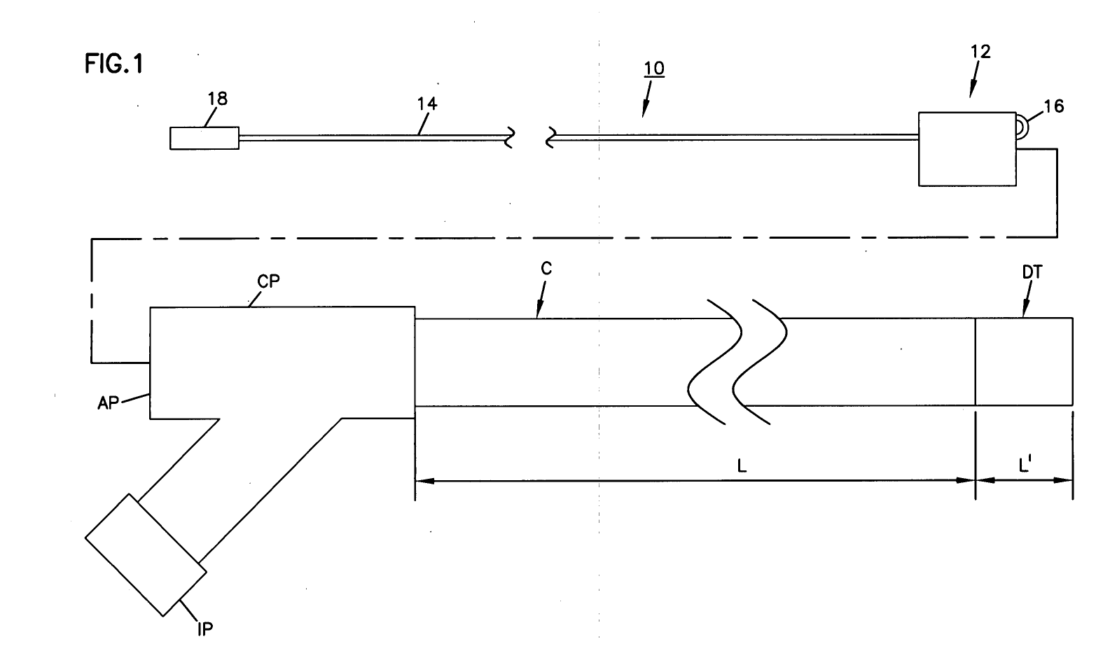

With initial reference to FIGS. 1-8, a first embodiment of the present invention will now be described. In FIG. 1, an occlusion system 10 is shown for use with a conventional prior art catheter C. This embodiment describes a convenient method to convert a guide catheter into an occlusion device.

Throughout the present description, the catheter C is described as the conventional guiding catheter of extruded polymer material. It will be appreciated the present invention is applicable to micro-catheters or other tubular instruments inserted within a patient's body.

The catheter C has a main body length of approximately 90 to 100 cm and terminating at a soft distal tip DT with a length L′ of 2 mm. A proximal end of the catheter C includes a prior art luer hub connected to a rotating hemostasis valve having a tool access port AP and an injection port IP. The access port AP is for admitting tools such as guide wires, micro catheters or the like into the internal lumen IL. The injection port IP is commonly used for pressure monitoring, for injection of contrast media or other fluids.

Catheters C come in many sizes with a typical length L being about 90 to 100 centimeters. The outside diameter of the catheter C can range from 5 to 9 French. The fore-going dimensions are representative of guide catheters. Micro-catheters are typically about 150 cm long with a diameter tapering from 3.5 to about 2 French. Guide wires are commonly ⅔ to 1.5 French.

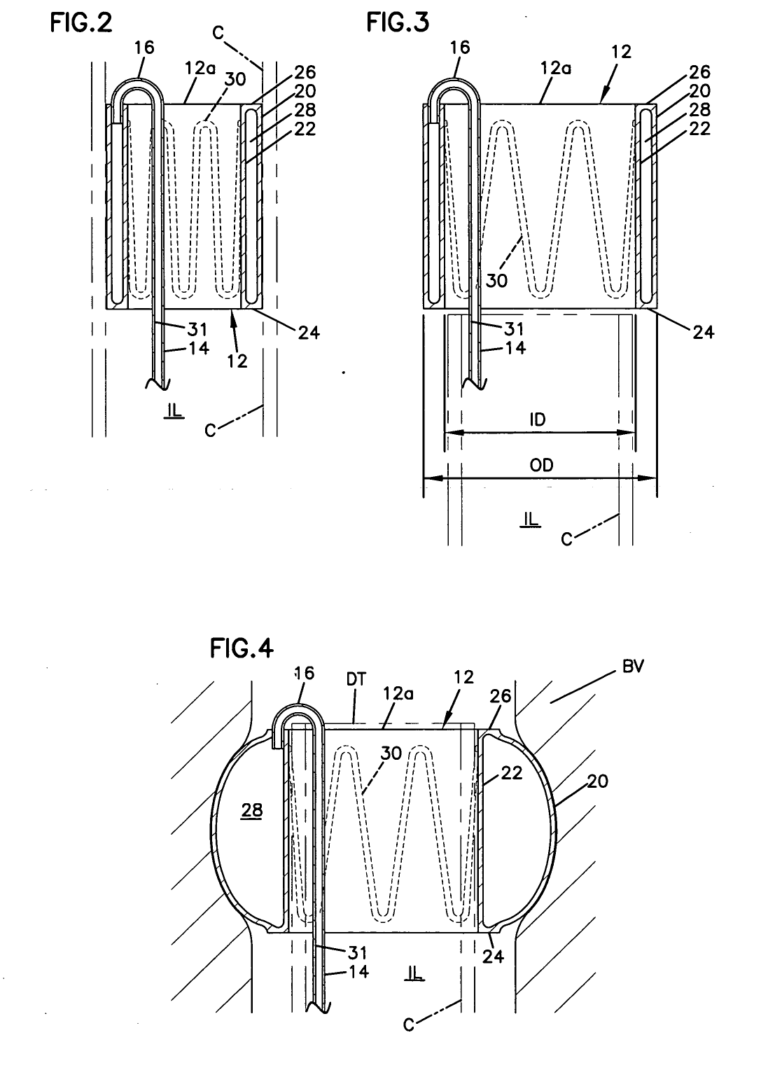

For purpose of illustration and not intended to limit the applicability of the present invention, a typical catheter C has a length L of 100 cm, outside diameter of 7 French and an internal lumen IL with an internal diameter commonly of about 0.073 inches. The internal lumen IL is not shown in FIG. 1 but is shown in FIGS. 2-8.

As previously described, the catheter C is advanced through the vasculature to a target location. For example, the catheter C can be advanced through the carotid artery of a patient until the distal tip DT attains positioning near the site to be treated or a branching artery. Prior art occlusion devices such as detachable balloons, micro catheters for use with coils or the like may be admitted through the access port AP and discharged from the soft flexible distal tip DT to the target location. The catheter C can also be used to pass a balloon catheter through the guide catheter C for further advancement to a desired location within the vasculature.

A clinical risk in advancing catheters such as guide catheters or micro catheters through the vasculature of the brain includes inadvertent perforation of the vessel by the catheter C. In such event, it is recognized to be desirable to occlude the blood vessel in a very rapid manner to prevent stroke or other serious adverse consequences. The desired occlusion blocks blood flow through the annular space defined by opposing surfaces of the micro catheter or guide catheter and the blood vessel. The occlusion system 10 of the present invention provides for such rapid deployment of an occlusion member in an emergency situation.

The occlusion system 10 includes an occlusion member 12 at a distal end of the occlusion system 10. An elongated advancing member 14 has a distal end 16 secured to the occlusion member 12. A proximal end of the advancing member 14 is provided with an inlet port 18 for injection of an inflation fluid as will be described.

With reference to FIGS. 2-4, the occlusion member 12 is shown in greater detail. The occlusion member 12 is a double-walled balloon preferably formed of silicone to have an outer wall 20 and an inner wall 22. Axial ends 24, 26 of the inner and outer walls 22, 20 are molded together to define a sealed chamber 28 positioned between the inner wall 22 and outer wall 20. A spring 30 is molded within the inner wall 22 and biased to urge the inner wall 22 to an expanded state illustrated in FIG. 3.

With reference to use with a typical 7 French catheter C as described above, in the expanded state of FIG. 3, the cylindrical occlusion member 12 has an outside diameter OD of about 0.117 inches (about 3.0 mm) and an inside diameter ID of about 0.095 inches (about 2.4 mm) defining an inner lumen 25. It will be appreciated that the specific sizing of a particular occlusion member 12 will vary for individually sized catheters as will be apparent to one of ordinary skill in the art.

The inner and outer walls 22, 20 are formed with a wall thickness of about 0.005 inches (about 0.13 millimeters). The spring 30 is preferably highly elastic material such as nickel-titanium alloy (more commonly known as nitinol) or other highly elastic material. The advancing member 14 is preferably a hypo tube structured of medical grade stainless steel material with an outer diameter OD′ of about 0.016 inches (about 0.41 millimeters) and an inner diameter ID′ of about 0.012 inches (about 0.30 millimeters).

The advancing member passes through the inner lumen 25. The distal end 16 of the advancing member 14 is curved and attached to the distal end 12A of the occlusion member 12. An interior lumen 31 of the advancing member 14 is in fluid flow communication with the sealed chamber 28.

As illustrated in FIG. 2, the occlusion member 12 may be contracted against the bias of spring 30 to a contracted state illustrated in FIG. 2 with the occlusion member 12 fully received within the internal lumen IL of the catheter C.

With the construction thus described, the use of the occlusion system 10 can now be described with reference to FIGS. 5-8.

In the event of an emergency situation requiring occlusion of the blood vessel BV in which the catheter C resides, the occlusion member 12 is urged against the bias of spring 30 to the contracted state of FIG. 2 and admitted through the access port AP into the lumen IL of the catheter C.

The physician advances the occlusion system 12 through the length L of the catheter C by pushing on the advancing member 14. The material of the advancing member 14 is sufficiently rigid to permit pushing on proximal end of the advancing member 14 to cause advancement of the distal end 16 of the advancing member 14. The distal end 16 pulls the occlusion member 12 throughout the length of the catheter C as illustrated in FIG. 5 in the direction of arrow A.

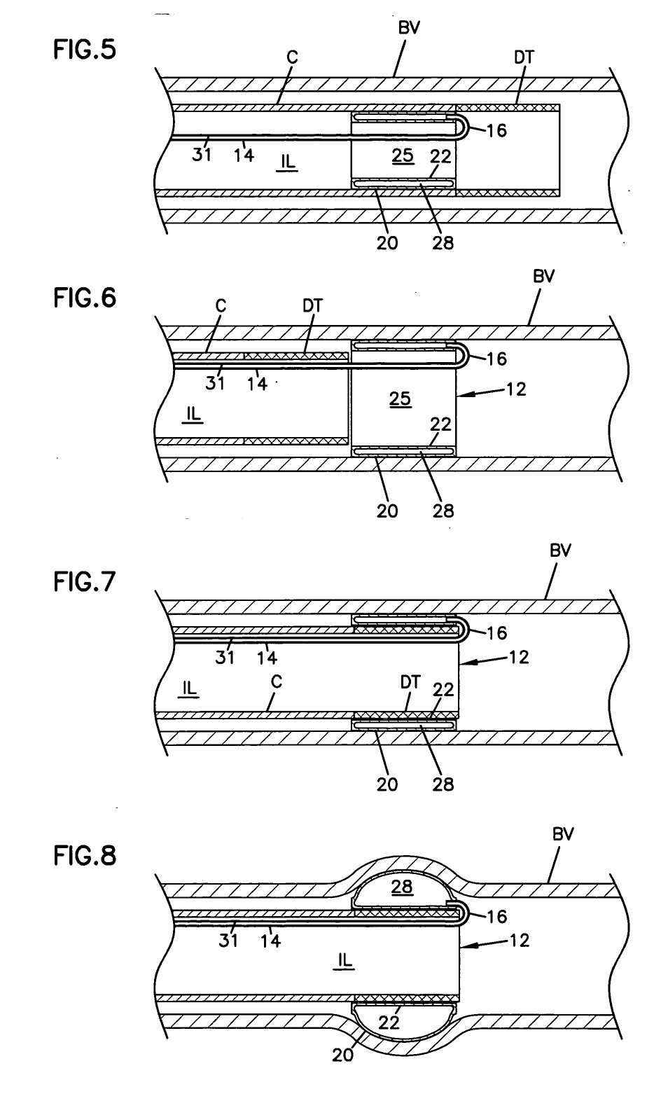

The occlusion system 10 is advanced throughout the entire length of the catheter C until the occlusion member 12 is advanced passed the distal tip DT as illustrated in FIG. 6. With the occlusion member 12 advanced past the distal tip DT, the occlusion member 12 expands to its expanded state of FIG. 3 by the urging of spring 30 until an inner diameter ID of the occlusion member is greater than the outer diameter of the catheter C.

With the occlusion member 12 expanded, the occlusion system 10 is retracted. The physician pulls on the advancing member 14 until the inner wall 22 of the occlusion member 12 surrounds the distal tip DT as illustrated in FIG. 7. With the occlusion member 12 so retracted, fluid (such as saline or the like) is admitted into the inlet port 18 and advanced throughout the lumen 31 of the advancing member 14 into the sealed chamber 28.

The fluid pressure in the chamber 28 causes the outer wall 20 to balloon radially outwardly as illustrated in FIGS. 4 and 8. The highly flexible inner wall 22 is restrained from ballooning inwardly by reason of the opposing material of the distal tip DT.

Inflation of the balloon of the occlusion member 12 causes the outer wall 20 to abut the opposing surfaces of the blood vessel BV. This restricts blood flow past the catheter C. In the event of a tear or perforation distal to the distal tip DT, in a cerebral artery, the occlusion member 12 prevents blood flow through the artery BV and out of the tear into the brain tissue. With the emergency occlusions so provided, the physician can then take necessary steps to repair the perforation or otherwise treat the patient.

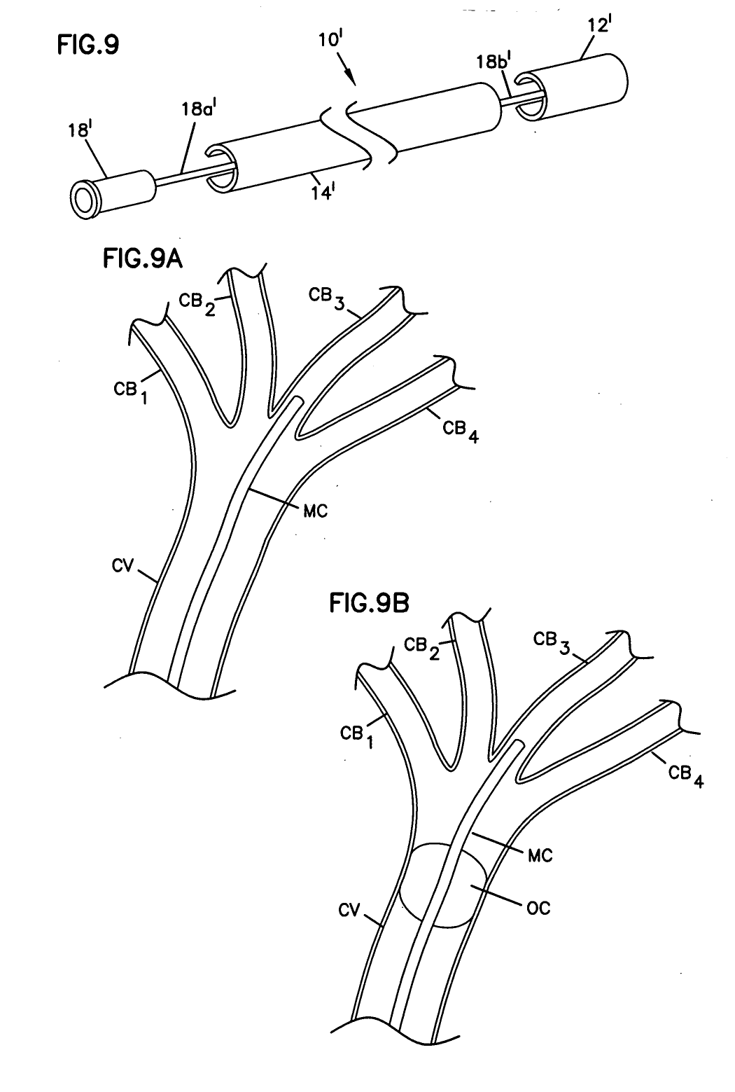

FIGS. 9-12A illustrate an alternative embodiment of the invention where an occlusion system 10′ is advanced along a pre-positioned catheter (as previously described) along the outer surface of the catheter rather than through the lumen of the catheter as in the embodiment of FIGS. 1-8. This design is particularly advantages where the catheter C is a microcatheter.

FIG. 9A illustrates the use of a microcatheter in a main cerebral vessel CV which branches into multiple cerebral vessel branches CB₁, CB₂, CB₃ and CB₄. The physician has advanced a distal tip DT of a microcatheter MC into the third branch CB₃ for delivery of a therapy (e.g., delivery of stent, coil or therapeutic agent). In this environment, hemodynamics (such as rate or turbulence of blood flow) may make such delivery difficult. A temporary occlusion proximal to the distal tip DT can interrupt blood flow until the therapy is delivered. Such an occlusion is shown as a balloon OC in the main branch CV in FIG. 9B. The invention of FIGS. 9-12A provides a convenient and rapid method for creating such occlusion when the need arises.

The occlusion system 10′ includes an occlusion member 12′ at a distal end of an advancing member 14′. At a proximal end of the system 10′, an inlet port 18′ is provided for admitting a pressurized fluid.

The advancing member 14′ is a split tube construction having an inner diameter approximate to the outer diameter of the catheter C. The advancing member 14′ is made of any flexible material which is elastically biased to a circular shape but which can be spread open along the split to be placed on the shaft of a catheter.

The advancing member 14′ has an inner lumen 31′ extending along its axial length as illustrated in FIG. 11. At the proximal end of the advancing member 14′ a tube 18a′ connects the inlet port 18′ with the lumen 31′.

The occlusion member 12′ is a double walled balloon having a plastic inner wall 22′ and a silicone outer wall 20′ sealed at their axially edges. Opposing surfaces of the walls 20′, 22′ define a sealed chamber 28′. A distal tubing 18b′ connects the sealed chamber 28′ with the lumen 31′.

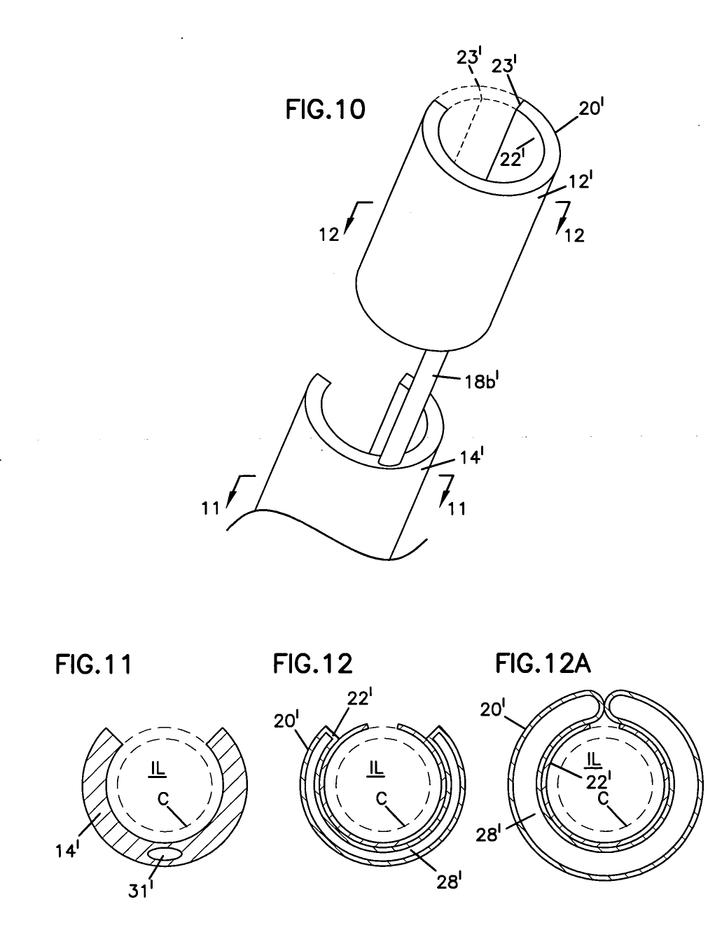

The inner wall 22′ is preferably formed of a plastic having sufficient rigidity to permit a balloon inflation without distorting the wall 22′. The wall 22′ has an axial slit 23′ along its axial length. The material of the wall 22′ is selected for the wall 22′ to be sufficiently flexible and elastic to permit it to be opened to be snapped onto the outer circumference of the catheter C. In a rest state, the slit 23′ is closed as illustrated in phantom lines in FIG. 10.

With the construction thus described, the occluding member 12′ may be snapped on to a guide catheter or other catheter and advanced along the length of the catheter by a physician pushing on the advancing member 14′. The axial rigidity of the advancing member 14′ and the tubes 18a′ and 18b′ transmit the advancing force to the occlusion member 12′ to advance it along the length of the catheter.

The occlusion member 12′ may be positioned at any point along the length of the catheter C. Fluid is then injected to the inlet port 18′ to inflate the outer wall 20′ as illustrated in FIG. 12A. The inflation of the balloon 20′ occludes the space between the catheter and the blood vessel. The system 10′ can be rapidly placed onto a catheter that has already been introduced into a blood vessel and rapidly advanced along the length of the blood vessel to a desired occlusion location.

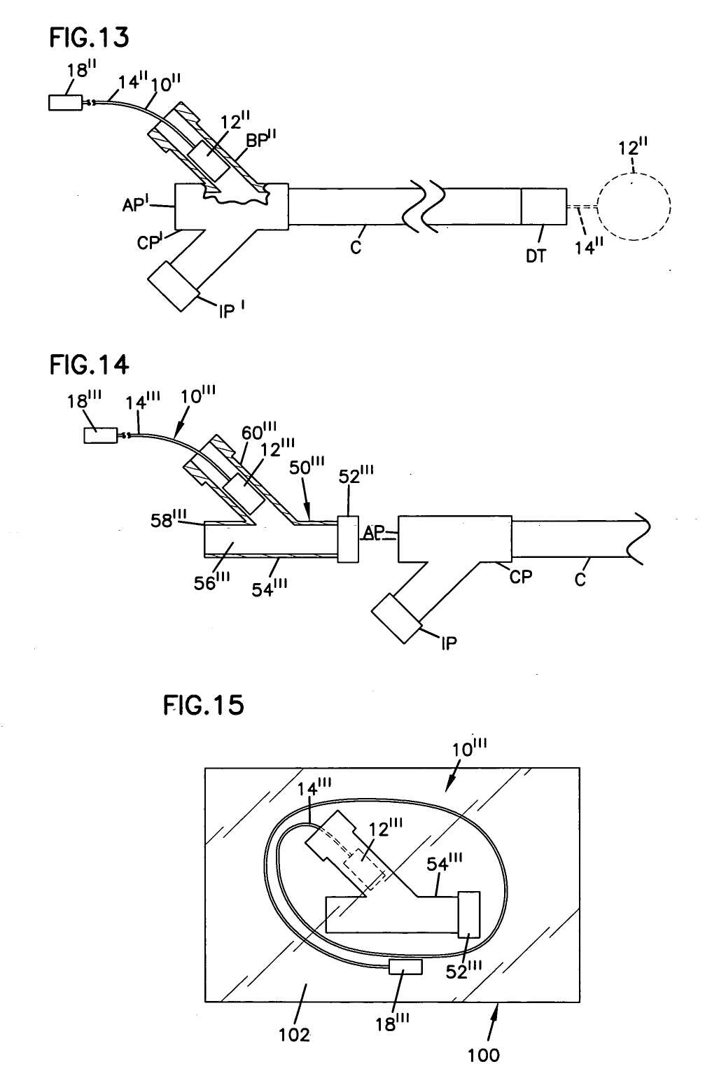

FIGS. 13-15 illustrate a still further embodiment of the present invention. In FIG. 13, the catheter C has a modified coupling port CP′ which, like the prior art, has an injection port IP′ and access port AP′. Differing from the prior art, the coupling port CP′ has an emergency balloon port BP′ preloaded with an occlusion system 10″. The occlusion system 10″ includes a micro balloon an occlusion member 12″ connected to an inlet port 18″ by a tubular advancing member 14″.

Unlike the annular balloons of the previously described embodiments, the balloon 12″ has no central lumen. Instead, in the event of a need for rapid occlusion, the advancing member 14″ is pushed distally by the physician to advance the balloon 12″ throughout the length of the catheter C and beyond the distal tip DT at which point fluid can be injected into the inlet port 18″ and fully inflate the balloon 12″ for complete occlusion of the blood vessel distally to the distal tip DT as illustrated in phantom lines in FIG. 13. With the embodiment of FIG. 13 the rapid deployment occlusion system 10′ is preloaded in a novel catheter design with a novel coupling port CP′.

FIGS. 14 and 15 illustrate an alternative to the embodiment of FIG. 13. In FIGS. 14 and 15, the occlusion system 10′″ is provided for attachment to a conventional catheter C having a conventional coupling port CP with convention access port AP and convention injection port IP.

The embodiment of FIG. 14 includes an accessory coupling 50′″ having a generally Y-shaped configuration and includes an attachment end 52′″ adapted for direct attachment to the access port AP. The accessory coupling 50′″ has an elongated cylindrical body 54′″ which extends in axial alignment with the access port AP when the attachment end 52′″ is attached to the access port AP. The main body 54′″ has a lumen 56′″ extending throughout its length and open at a proximal end 58′″ for insertion of tools, guide wires, micro catheters or other devices which the physician would normally desire to place through the access port AP during an interventional procedure.

Extending at an acute angle to the main body 54″′ is an occlusion port 60″′ containing a balloon 12′″ having an advancing member 14′′ and an inlet port 18′″as referenced in the embodiment of FIG. 13.

Accordingly, after attachment of end 52′″ to the access port AP, the end 58′′ serves as the access port for the coupled assembly permitting the physician to use the catheter C in a conventional manner and to admit accessory tools such as guide wires, micro catheters or the like through the inlet 58′″ and through the access port AP and into the catheter. In the event of a perforation, the occlusion balloon 12′″ is advanced through the catheter as described with reference to FIG. 13.

With the assembly described, no time is wasted in providing the occlusion in an emergency situation.

The occlusion member 10′″ may be individually packaged in a sealed, sterile package 100 illustrated in FIG. 15 which may include an opaque backing 102 with a clear cover 104 with the contents sterilized through any conventional manner acceptable to regulatory authorities such as the United States Food and Drug Administration. Such sterilization techniques are readily known to those skilled in the art.

With the invention of FIGS. 14 and 15, when a physician is performing an interventional procedure with a catheter where it is believed that risk of perforation may exist, the surgeon may, in advance of such procedure, open the package 100 and attach the occlusion system 10′″ to the conventional port CP so that emergency occlusion is instantly available in the event of an unintended perforation.

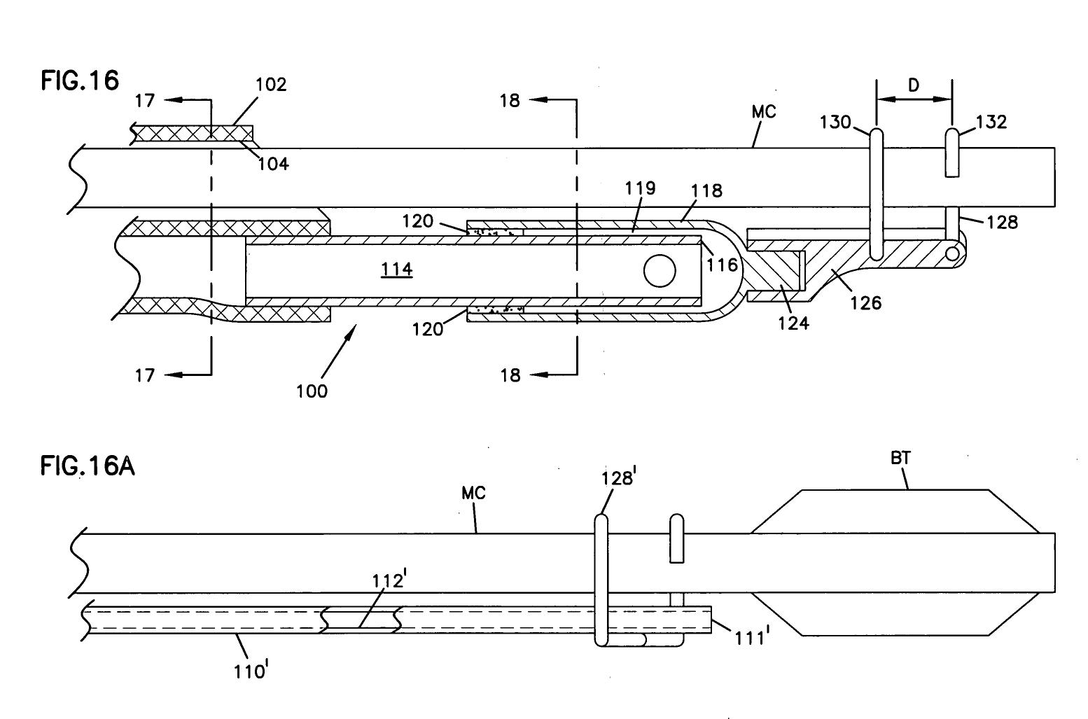

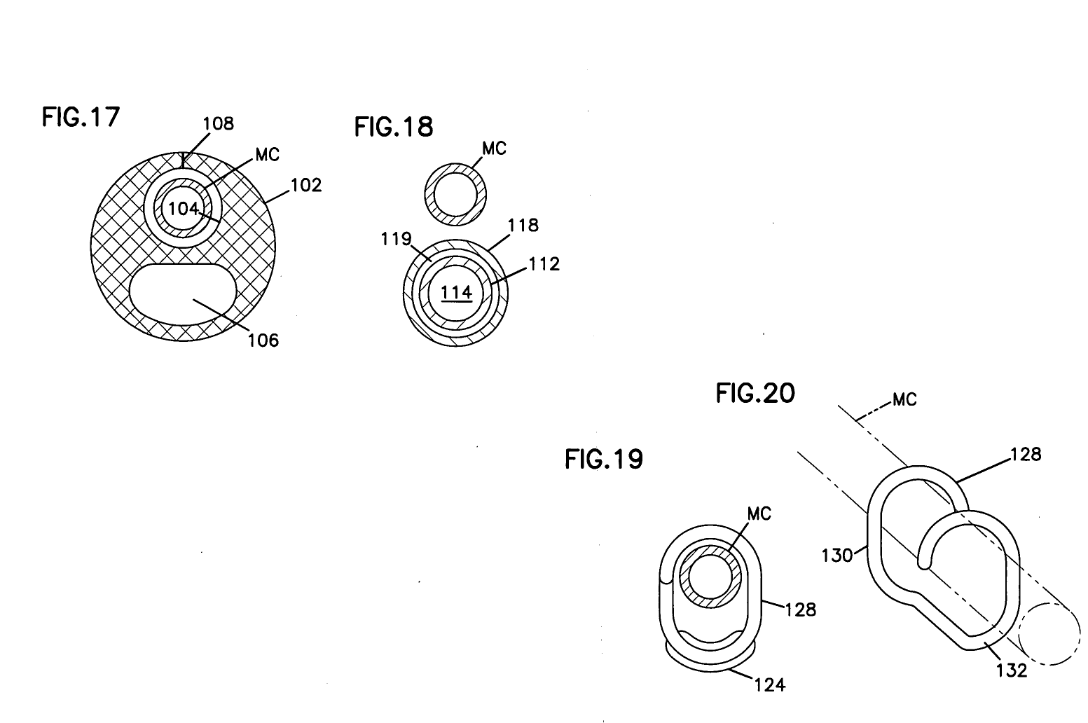

In FIG. 16, a balloon delivery system 100 is shown deployed on a micro catheter MC. The balloon delivery system includes a tubular body 102 having a main lumen 104 and an inflation lumen 106.

A slit 108 is formed through the main tubular body 102 in communication with the main lumen 104. Accordingly, the tubular body 102 may be open at the slit 108 and placed around the external diameter of the micro catheter MC with the micro catheter MC residing within the main lumen 104.

The tubular body 102 terminates at a distal end 110. A tube 112 with internal lumen 114 is bonded to the distal end of the inflation lumen 106. The tube 112 terminates at a distal end 116.

The distal end 116 of the tube 112 is surrounded by a silicone balloon 118. The balloon 118 has its proximal end bonded to the tube 112 by adhesive 120. A side port 122 is formed in the tube 112 to admit inflation fluid into the space defined between the balloon 118 and the tube 112.

The balloon 118 has a distal hub 124 bonded to a silicone shuttle 126. The shuttle 126 is bonded to a metal clip 128 having two windings 131, 132 such that the clip 130, 132 can be mounted by snapping it on to a micro catheter MC. The clip 128 may be formed of any flexible material such as thin stainless steel or nitinol. While the balloon 118 and the silicone shuttle 126 are shown as separate elements bonded by adhesive or otherwise, they could be molded as a single piece.

With the apparatus thus described, a delivery system 100 can be mounted on a catheter such as a micro catheter MC by snapping the clip 128 around the outside diameter of the micro catheter and by opening the main body 102 at the slit 108 to receive the body of the micro catheter within the main lumen 104. The delivery system 100 can then be advanced along the length of the micro catheter until the balloon 118 is in the region of an area of desired occlusion. At such point, fluid can be admitted to the lumen 106 which passes into the space between the tube 112 and the balloon 118 to inflate the balloon 118 and create a desired occlusion.

FIG. 16A illustrates an alternative embodiment to FIG. 6. In FIG. 16A the microcatheter MC is shown as having a balloon tip BT for purpose of illustrating the versatility of the present invention. The microcatheter could be identical to the microcatheter MC in FIG. 16 and without a balloon tip BT.

In the embodiment of FIG. 16A, the shuttle 126 is mounted to a hollow catheter 110′ having an internal lumen 112′ extending throughout its length. The distal tip 111′ of the catheter 110′ extends beyond the shuttle and is affixed thereto. With this embodiment, after placing the original microcatheter MC′, the physician may find it desirable top have an additional lumen in the blood vessel. The hollow catheter 110′ can be snapped onto the microcatheter MC′ by snapping the shuttle onto the microcatheter MC′ and using the microcatheter MC′ as a guide to deliver the second catheter 110′ to a desired position in the blood vessel. The newly delivered catheter 110′ can then be used to deliver therapeutic devices or agents or contract media.

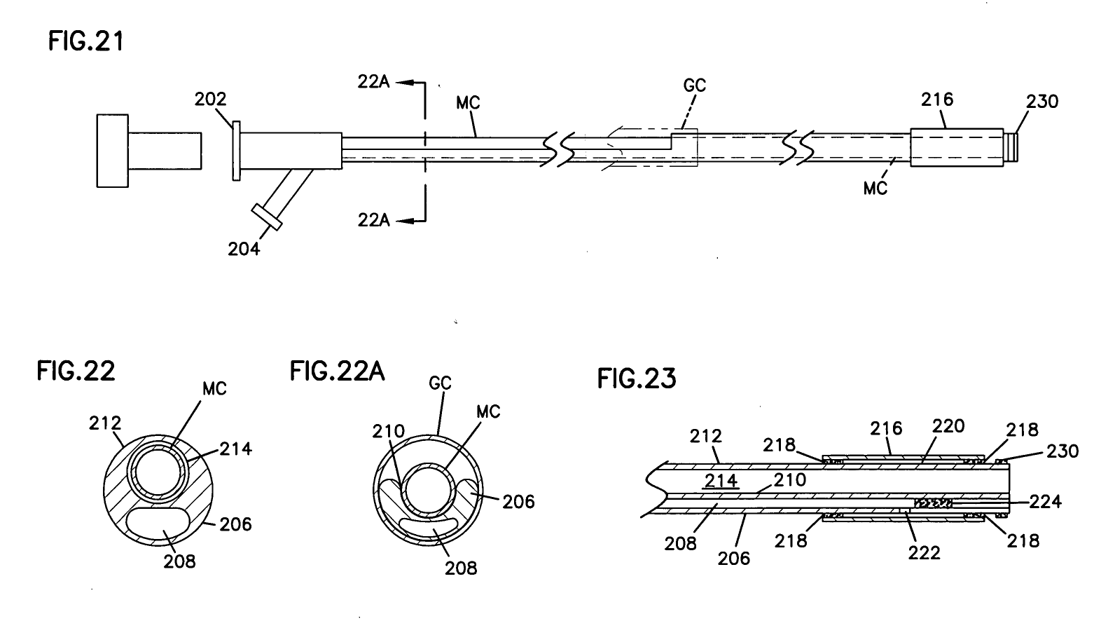

FIG. 21 is a side elevation view of a still further embodiment of the present invention. In FIG. 21, a delivery system 200 is shown having a distal end of length LD and a proximal length of L_{P}.

The proximal portion L_{P} is connected to an adapter having a wire port 202 and an inflation port 204. The proximal length Lₚ is a tube 206 having a lumen 208 and a concave surface 210 to receive a micro catheter MC. The distal portion LD is an extension of the tubular portion 206 and includes a portion 212 which defines a main body lumen 214 sized to receive the micro catheter MC.

At its distal end, a silicone balloon 216 surrounds the body portion 212. The balloon 216 is a cylinder of silicone as previously described and bonded to the external surfaces of the body portions 212, 206 by adhesive 218. The adhesives also act to define an internal balloon lumen 220 defined between the opposing surfaces of the silicone 216 and the surfaces 212, 206.

A port 222 is formed through the sidewall of tube 206 into communication with inflation lumen 208. The port 222 is positioned in fluid flow communication with the lumen 220 so that inflation fluid can flow from the lumen 208 into the lumen 220 of the balloon to inflate the balloon 216. An adhesive plug 224 is placed within the lumen 206 distal to the port 222 to seal the lumen 206 distally.

FIG. 1 also shows a well-known toughy adapter 203 to be inserted within the wire port 202 to crimp a guide wire (not shown) and hold the wire accurately positioned. The guide wire is passed through the wire port 202 is admitted to the lumen 206. The guide wire can then be removed from the lumen when it is desired to admit inflation fluid into the lumen 208.

The proximal length L_{P} is sized to have a length to reside within the length of a guide wire through which a micro catheter MC is placed. The distal length LD is in practice approximately 30-40 centimeters long to track a micro catheter and advance the balloon 216 to a desired occlusion site.

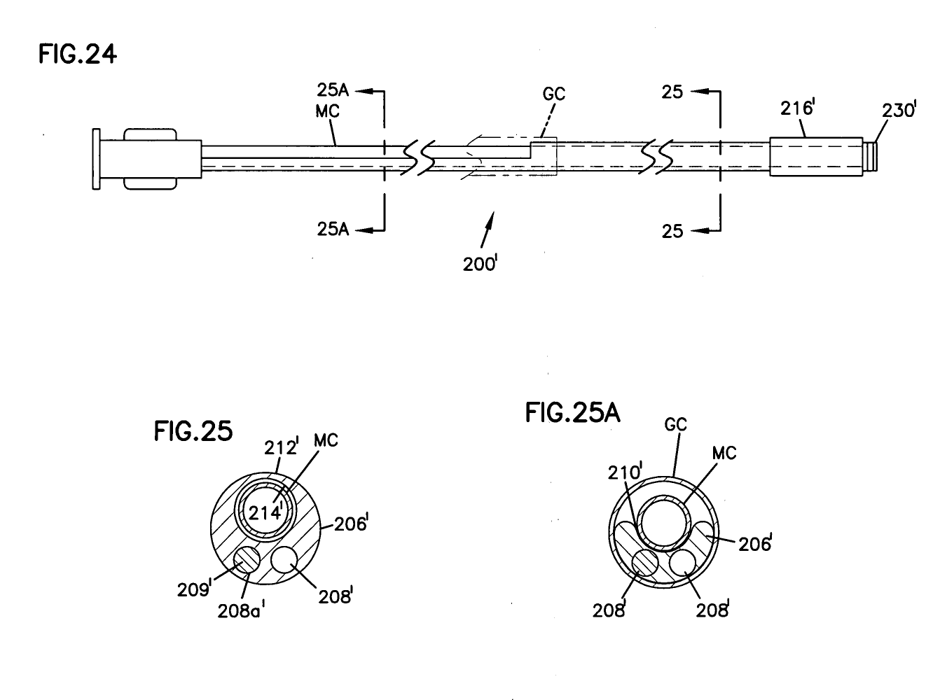

FIGS. 24 and 25 illustrate an alternative embodiment of that of FIGS. 21-23. Elements of FIGS. 24, 25 and 25A are numbered identically to those of FIGS. 21-23 with the addition of an apostrophe to distinguish the embodiments. In both of FIGS. 21 and 22 an x-ray marker 230 is provided at distal end of the balloon to permit fluoroscopic recognition of the placement of the balloon. In the embodiments of FIGS. 24 and 25, a wire lumen 208a′ is provided within the main body. A wire 209 is permanently placed within the lumen 208a′ to add stiffening to the apparatus 200′ along its length to aid in advancement of the delivery system to a desired location.

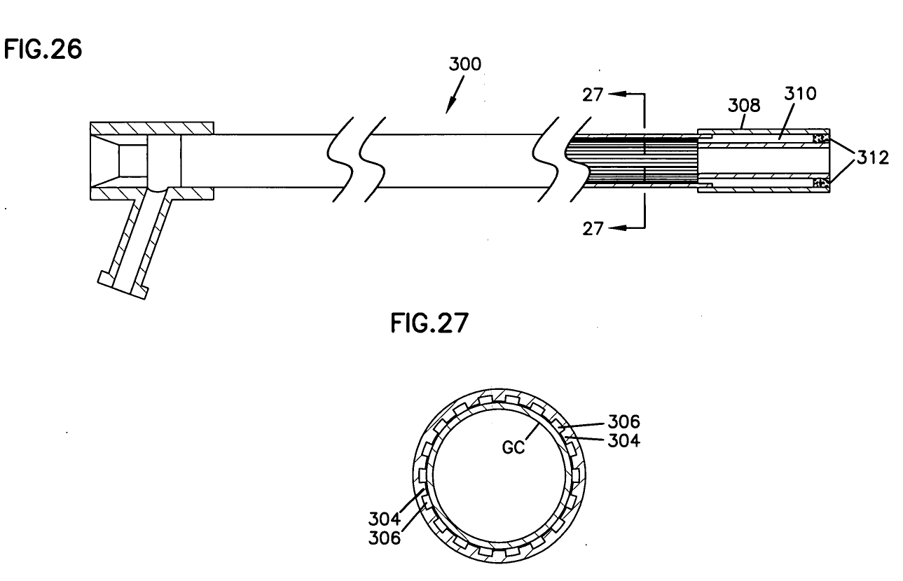

FIGS. 26 and 27 illustrate a still further embodiment of the present invention where delivery system 300 is positioned over a guide catheter GC (shown only in cross-section in FIG. 27). The delivery system 300 includes a main body portion 302 having a plurality of splines 304 on its internal surface. The splines 304 oppose an external surface of the guide catheter GC to define a plurality of individual lumens 306 for delivery of an inflation fluid. The lumens 306 are in fluid flow communication with the interior of a balloon 308 having a lumen 310. The ends of the lumen 310 are sealed by adhesives 312.

Having disclosed the forgoing concepts in a preferred embodiment, it will be appreciated that modifications and equivalents may occur to one of ordinary skill in the art. It is intended that such modifications and equivalents be included within the scope of the claims which are appended hereto. It will be recognized that the devices described in the present application may be provided with radial opaque markers at any convenient point along their length or at the distal tip of the occlusion devices to permit visualization under fluoroscopy. Elements may be coated with any lubricious coating known in the art to facilitate smooth advancement of the occlusion members along or through the catheter.

We claim:

1. An occlusion system for use with a catheter having a catheter proximal end and a catheter distal end, said catheter further having a catheter wall with an outer catheter dimension at said catheter distal end and with said catheter wall defining a catheter lumen with a longitudinal axis extending from said catheter proximal end and through said catheter distal end, said occlusion system comprising:
an occlusion member having an inner wall and an outer wall, said inner wall defining an occlusion member lumen extending throughout an axial length of said occlusion member;
said outer wall expandable between a contracted state with a first transverse dimension and an expanded state with a second transverse dimension, said second transverse dimension greater than said outer catheter dimension;
said occlusion member sized for said occlusion member to be received at least partially around an exterior of said catheter lumen with said catheter received within said occlusion member lumen and advanced from said catheter proximal end toward said catheter distal end.

2. An occlusion system according to claim 1 further comprising an actuator for urging said occlusion member from said contracted state to said expanded state.

3. An occlusion system according to claim 2 wherein said outer wall and inner wall are separated by a sealed chamber and said actuator includes an actuator lumen for admitting a fluid under pressure into said chamber.

4. An occlusion member according to claim 3 wherein said outer wall and inner wall are walls of a double-walled balloon sealed at proximal and distal axial edges.

5. An occlusion system according to claim 1 further comprising:
an advancing member having an advancing member proximal end secured to said occlusion member and an advancing member distal end;
said advancing member proximal and distal ends connected by an advancing member body having a rigidity for said advancing member proximal end to urge said occlusion member through said catheter lumen upon application of an axial force to said advancing member proximal end;
said actuator lumen extending through said advancing member.
